(19) United States
(12) Patent Application Publication
(10) Pub. No.: US 2026/0201101 A1
(43) Pub. Date: Jul. 16, 2026

(54) MODIFIED CYSTAMINE CURING AGENT AND PREPARATION METHOD THEREFOR, AND SELF-REPAIRING POLYURETHANE AND PREPARATION METHOD THEREFOR

(71) Applicant: AEROSPACE RESEARCH INSTITUTE OF MATERIALS & PROCESSING TECHNOLOGY, BEIJING (CN)

(72) Inventors: YANG ZHANG, BEIJING (CN); NIJUAN SUN, BEIJING (CN); YUQI ZHOU, BEIJING (CN); WENPENG DUAN, BEIJING (CN); YONG XU, BEIJING (CN); CHUNYAN SUN, BEIJING (CN); BO TANG, BEIJING (CN)

(21) Appl. No.: 19/136,437

(22) PCT Filed: Oct. 27, 2023
(86) PCT No.: PCT/CN2023/126980

§ 371 Date: Jun. 6, 2025

(30) Foreign Application Priority Data
Dec. 6, 2022 (CN) 202211558478.7

(51) Int. Cl. C08G 18/38; C07C 319/22; C08G 18/10; C08G 18/28

(52) U.S. Cl. CPC C08G 18/3868; C07C 319/22; C08G 18/10; C08G 18/2845

(57) ABSTRACT

A method for preparing a modified cystamine curing agent is provided. The method includes: obtaining a reaction solution by reacting an aqueous solution of cystamine dihydrochloride with an aqueous solution of KOH; extracting the reaction solution with a dichloromethane solution, followed by rotary evaporating and drying to obtain cystamine; and obtaining an acrylamide-modified cystamine curing agent by reacting the cystamine with acrylamide.

### CROSS-REFERENCE TO RELATED APPLICATIONS

This application is a U.S. national stage application of International Application No. PCT/CN2023/126980 filed Oct. 27, 2023, which is based upon and claims priority to Chinese Patent Application 202211558478.7, filed on Dec. 6, 2022, the entire contents of which are incorporated herein by reference.

### TECHNICAL FIELD

The present disclosure belongs to the technical field of intelligent materials, and relates to a modified cystamine curing agent and a preparation method thereof, and a self-healing polyurethane and a preparation method thereof.

### BACKGROUND

A self-healing polyurethane prepared based on dynamic covalent bonds has a potential for self-healing under mild conditions, which may, when a material is damaged, heal a physical damage of the material and extend a service life of the material through an exchange mechanism of dynamic covalent bonds. The self-healing polyurethane is a smart material with many applications in damping, 3D printing, bionic materials and other fields.

The dynamic covalent bonds include disulfide bonds, Diels-Alder, Schiff bases, etc. The disulfide bond (also known as a disulfide linkage) has a low bond energy and mild self-healing conditions, and may respond to a variety of environmental stimuli (such as a temperature, alkaline conditions, a light, etc.), which not only provides a possibility of self-heal at room temperature, but also may achieve the self-heal in response to a variety of environmental factors. The disulfide bond is a reversible bond with great application prospects. A self-healing efficiency of the self-healing polyurethane based on a disulfide bond mechanism is generally very high.

There have been reports on a preparation of polyurethane with a self-healing function by reacting chain extenders containing the disulfide bonds with polyurethane raw materials. Aliphatic amines containing the disulfide bonds have a great application potential as curing agents for the preparation of self-healing polyurethane due to their designable structures. Currently, there have been many reports on the self-healing polyurethane with a good mechanical strength and a high self-healing efficiency. However, a reaction between the aliphatic amine containing the disulfide bond and polyurethane prepolymer releases a lot of heat, which is difficult to control. Therefore, most methods for preparing the self-healing polyurethane use a solvent method. A polyurethane elastomer prepared by the solvent method is not only difficult to remove a solvent clearly and easily produces many bubble defects, but also pollutes the environment, which is not user-friendly, and cannot be industrially produced.

### SUMMARY

The present disclosure provides the following technical solutions.

A method for preparing a modified cystamine curing agent includes:
obtaining a reaction solution by reacting an aqueous solution of cystamine dihydrochloride with an aqueous solution of KOH;
extracting the reaction solution with a dichloromethane solution, followed by rotary evaporating and drying to obtain cystamine; and
obtaining an acrylamide-modified cystamine curing agent by reacting the cystamine with acrylamide.

A method for preparing a self-healing polyurethane includes:
obtaining an isocyanate-terminated polyurethane prepolymer by reacting diisocyanate with a dihydric alcohol;
obtaining an epoxy-terminated polyurethane prepolymer by reacting the isocyanate-terminated polyurethane prepolymer with glycidol; and
obtaining a solvent-free self-healing polyurethane based on dynamic disulfide bonds by reacting the epoxy-terminated polyurethane prepolymer with the above modified cystamine curing agent.

### BRIEF DESCRIPTION OF THE DRAWINGS

FIG. 1 is a Fourier transform infrared spectrum of a curing agent obtained in Example 2 of the present disclosure.

### DETAILED DESCRIPTION

The following detailed description of the present disclosure may make features and advantages of the present disclosure more clear and explicit.

The word “explanatory” is used exclusively herein to mean “serving as an example, embodiment, or illustration”. Any embodiment described herein as “explanatory” is not necessarily to be construed as preferred or advantageous over other embodiments. While various aspects of embodiments are shown in drawings, the drawings are not necessarily drawn to scale unless otherwise specified.

The purpose of the present disclosure is to overcome the above defects, provide a modified cystamine curing agent and a preparation method, a self-healing polyurethane and a preparation method, and solve technical problems that a solvent is difficult to remove and bubbles are easily generated in a process of generating a self-healing polyurethane by a solvent method. The present disclosure realize a solvent-free preparation of a self-healing polyurethane elastomer based on dynamic disulfide bonds by reacting an epoxy-terminated polyurethane prepolymer with an aliphatic amine containing a disulfide bond. An obtained self-healing polyurethane has not only good toughness but also good self-healing performance.

In order to achieve the above purpose, the present disclosure provides the following technical solutions.

A method for preparing a modified cystamine curing agent includes:
obtaining a reaction solution by reacting an aqueous solution of cystamine dihydrochloride with an aqueous solution of KOH;
extracting the reaction solution with a dichloromethane solution, followed by rotary evaporating and drying to obtain cystamine; and
obtaining an acrylamide-modified cystamine curing agent by reacting the cystamine with acrylamide.

Further, the cystamine is reacted with the acrylamide at a reaction temperature of 60 to 100° C. for a reaction time of 4 to 10 h, and a molar ratio of the cystamine to the acrylamide is 1.0:1.5 to 1.0:2.1.

A modified cystamine curing agent is obtained by the above method for preparing the modified cystamine curing agent.

A method for preparing a self-healing polyurethane includes:
obtaining an isocyanate-terminated polyurethane prepolymer by reacting diisocyanate with a dihydric alcohol;
obtaining an epoxy-terminated polyurethane prepolymer by reacting the isocyanate-terminated polyurethane prepolymer with glycidol; and
obtaining a solvent-free self-healing polyurethane based on dynamic disulfide bonds by reacting the epoxy-terminated polyurethane prepolymer with the above modified cystamine curing agent.

Further, the diisocyanate includes one or more of toluene diisocyanate TDI, isophorone diisocyanate IPDI, methylene diphenyl diisocyanate MDI, dicyclohexylmethane diisocyanate HMDI or hexamethylene diisocyanate HDI.

The dihydric alcohol includes one or more of polycaprolactone diol PCL, polypropylene glycol PPG, polyethylene glycol PEG, or polytetramethylene glycol PTMG.

The diisocyanate is reacted with the dihydric alcohol in a molar ratio of the diisocyanate to the dihydric alcohol of 3.0:2.0 to 5.0:2.0.

Further, reacting the diisocyanate with the dihydric alcohol includes:
dehydrating the dihydric alcohol by vacuuming at a temperature of 100 to 120° C. for a vacuuming time of 2 to 5 h;
cooling dehydrated dihydric alcohol to a temperature below 60° C.; and
adding diisocyanate to cooled dihydric alcohol for reaction under stirring at 60 to 85° C. for 3 to 6 h under nitrogen protection to obtain the isocyanate-terminated polyurethane prepolymer.

Further, the isocyanate-terminated polyurethane prepolymer is reacted with glycidol at a reaction temperature of 50 to 85° C. for a reaction time of 2 to 6 h in a nitrogen atmosphere; and a molar ratio of the isocyanate-terminated polyurethane prepolymer to glycidol is 1.0:2.0 to 1.0:2.1.

Further, a molar ratio of a reactive functional group of the epoxy-terminated polyurethane prepolymer to a reactive functional group of the modified cystamine curing agent is 1.0:0.95 to 1.0:1.05; and
the reactive functional group of the epoxy-terminated polyurethane prepolymer is active hydrogen, and the reactive functional group of the modified cystamine curing agent is epoxy group.

Further, the epoxy-terminated polyurethane prepolymer is reacted with the modified cystamine curing agent at a reaction temperature of 80 to 130° C. for a reaction time of 6 to 24 h.

A self-healing polyurethane is obtained by the above method for preparing the self-healing polyurethane.

Compared with the related art, the present disclosure has at least one of the following beneficial effects.
(1) The present disclosure creatively proposes the acrylamide-modified cystamine curing agent, which is used to introduce a large number of amide bonds into a main chain structure of polyurethane. The amide bonds may form a large number of hydrogen bonds with carbamate bonds in a system, thereby synergizing with the disulfide bonds to enhance the self-healing performance of the polyurethane elastomer.
(2) The present disclosure creatively proposes the method for preparing the self-healing polyurethane, which does not require a solvent. By forming the epoxy-terminated polyurethane prepolymer, the reactivity of the prepolymer and the amine curing agent is reduced, and the reactivity of the epoxy-terminated polyurethane prepolymer and the modified cystamine curing agent is moderate, which is conducive to a discharge of bubbles during the curing process, and an obtained polyurethane material has fewer defects.
(3) The present disclosure may prepare curing agents with different amine values by adjusting the molar ratio of the acrylamide to the cystamine, and coordinate with the epoxy-terminated polyurethane prepolymer to adjust the mechanical performance of the polyurethane elastomer.

One aspect of the present disclosure provides a method for preparing an acrylamide-modified cystamine curing agent, which includes:
1) mixing an aqueous solution of cystamine dihydrochloride with an aqueous solution of KOH for reaction, extracting with a dichloromethane solution, and rotary evaporating and drying to obtain cystamine; and
2) obtaining an acrylamide-modified cystamine curing agent by reacting the cystamine obtained in the step 1) with acrylamide.

In the step 2) of the above method, a molar ratio of the cystamine to the acrylamide is 1.0:1.5 to 1.0:2.1; a reaction temperature is 60 to 100° C., preferably 75° C.; and a reaction time is 4 to 10 h.

Another aspect of the present disclosure provides a method for preparing a solvent-free self-healing polyurethane based on dynamic disulfide bonds, which includes:
1) obtaining an isocyanate-terminated polyurethane prepolymer by reacting diisocyanate with a dihydric alcohol;
2) obtaining an epoxy-terminated polyurethane prepolymer by reacting the isocyanate-terminated polyurethane prepolymer with glycidol; and
3) obtaining the solvent-free self-healing polyurethane based on the dynamic disulfide bonds by reacting the epoxy-terminated polyurethane prepolymer with the aforementioned acrylamide-modified cystamine curing agent prepared by the present disclosure.

In the step 1) of the above method, the diisocyanate is selected from at least one of toluene diisocyanate (TDI), isophorone diisocyanate (IPDI), methylene diphenyl diisocyanate (MDI), dicyclohexylmethane diisocyanate (HMDI) or hexamethylene diisocyanate (HDI).

The dihydric alcohol is selected from at least one of polycaprolactone diol (PCL), polypropylene glycol (PPG), polyethylene glycol (PEG) or polytetramethylene glycol (PTMG).

A molar ratio of the diisocyanate to the dihydric alcohol is 3.0:2.0 to 5.0:2.0.

The step 1) of the above method specifically includes:
dehydrating the dihydric alcohol by vacuuming at 100 to 120° C. for 2 to 5 h, then cooling to a temperature below 60° C., and adding diisocyanate for reaction under stirring at 60 to 85° C. for 3 to 6 h under nitrogen protection to obtain the isocyanate-terminated polyurethane prepolymer.

In the step 2) of the above method, a molar ratio of the isocyanate-terminated polyurethane prepolymer to glycidol is 1.0:2.0 to 1.0:2.1.

In the step 2) of the above method, a reaction temperature is 50 to 85° C., preferably 70° C.; a reaction time is 2 to 6 h; and a reaction atmosphere is a nitrogen atmosphere.

In the step 3) of the above method, a molar ratio of a reactive functional group of the epoxy-terminated polyurethane prepolymer to a reactive functional group of the acrylamide-modified cystamine curing agent (active hydrogen: epoxy group) is 1.0:0.95 to 1.0:1.05.

In the step 3) of the above method, a reaction temperature is 80 to 130° C., preferably 100° C.; and a reaction time is 6 to 24 h.

In addition, the solvent-free self-healing polyurethane based on the dynamic disulfide bonds prepared according to the above methods also belongs to the scope of protection of the present disclosure.

The acrylamide-modified cystamine (AMCY) curing agent of the present disclosure has a following reaction mechanism:

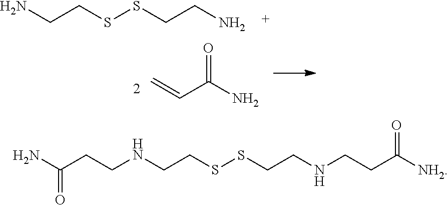

For the self-healing polyurethane based on the dynamic disulfide bonds, part of its reaction mechanism is:

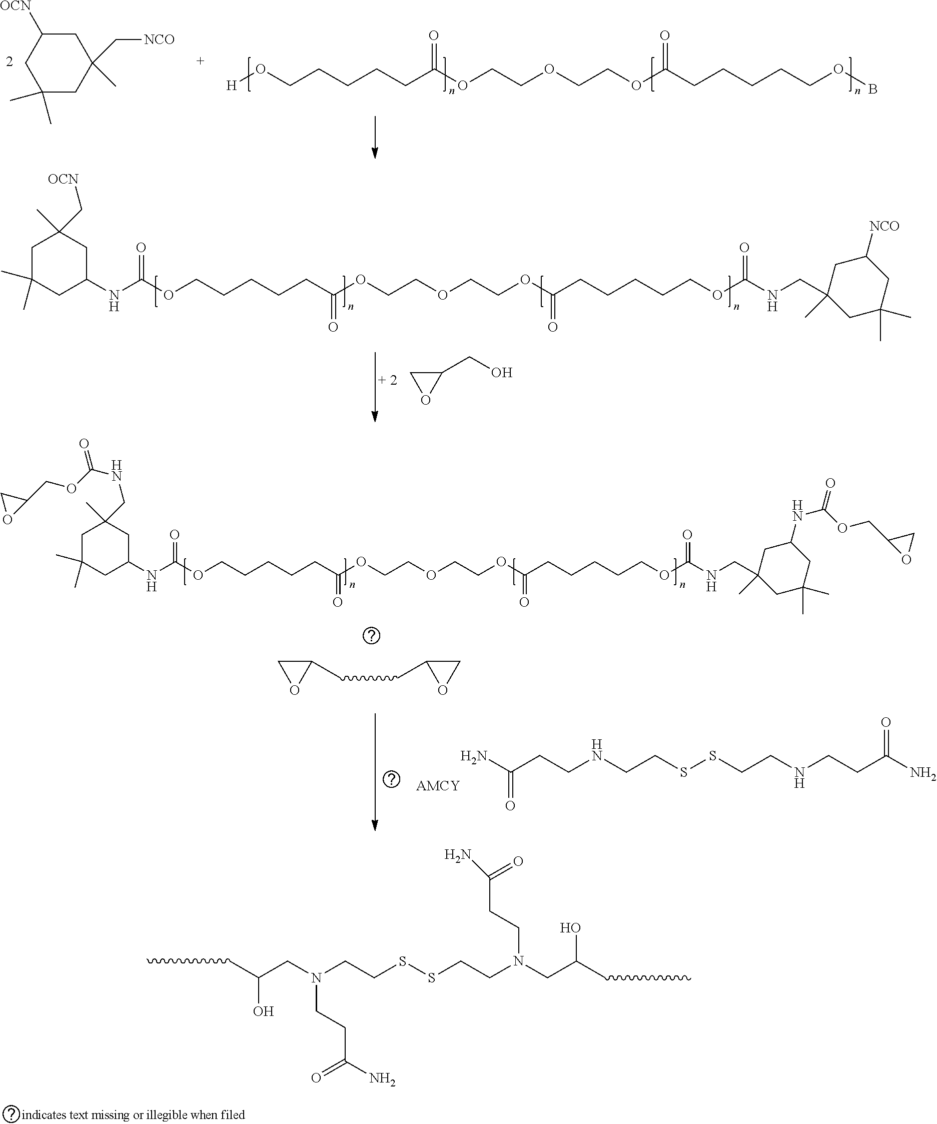

The present disclosure firstly uses the acrylamide to react with the cystamine to obtain the modified cystamine curing agent (the acrylamide-modified cystamine or a mixture of the acrylamide-modified cystamine and the cystamine). The reason for selecting the cystamine as a modification object is that its main chain structure contains the disulfide bond and it is an aliphatic primary amine with two active hydrogens on an end group. On the one hand, the disulfide bond may be introduced into the curing agent, and on the other hand, one of the active hydrogens therein may be replaced to introduce a hydrogen bond on a side group. Therefore, compared with a general aliphatic amine curing agent, the cystamine curing agent modified by the acrylamide contains the disulfide bond on the main chain of the structure and contains a hydrogen bond on the side chain of the structure. Both the disulfide bond and the hydrogen bond belong to the dynamic covalent bonds. Therefore, this type of curing agent is used as a curing component of the polyurethane, which may effectively improve the self-healing performance of polyurethane materials. The present disclosure uses the diisocyanate and the dihydric alcohol to prepare the polyurethane prepolymer, then uses the glycidol to end-cap the polyurethane prepolymer to obtain the epoxy-terminated polyurethane prepolymer. Generally, the polyurethane prepolymer end-capped with isocyanate has a relatively high activity, and a reaction with the aforementioned amine chain extender must be performed in a solvent. Therefore, the glycidol is used here to end-cap the polyurethane prepolymer, and after an original isocyanate group is replaced by an epoxy group, an activity is reduced, and curing under solvent-free conditions may be achieved. The curing agent and the prepolymer are reacted in a certain ratio to obtain the self-healing polyurethane.

In summary, the acrylamide-modified cystamine curing agent used in the present disclosure may introduce the disulfide bonds and a large number of hydrogen bonds into the main chain structure of the polyurethane, thereby improving the self-healing performance of the polyurethane elastomer, while the epoxy-terminated polyurethane prepolymer may reduce an activity of the curing reaction, thereby realizing the solvent-free method for preparing the self-healing polyurethane elastomer.

### Example 1

This Example provides an acrylamide-modified cystamine curing agent, which was prepared by a method including the following steps:
(1) mixing an aqueous solution of cystamine dihydrochloride with an aqueous solution of KOH for reaction;
(2) extracting with a dichloromethane solution, followed by rotary evaporating and drying to obtain 15.272 g of cystamine in an organic phase, with a yield of an actual product mass/a theoretical product mass=15.272/19.881=76.82%; and
(3) adding a certain amount of cystamine to a three-necked flask, and adding acrylamide in a stoichiometric ratio of the cystamine to the acrylamide of 1.0:2.1, heating to 75° C. and performing reaction for 10 h to obtain the modified cystamine curing agent, recorded as curing agent 1.

### Example 2

This Example provides an acrylamide-modified cystamine curing agent, which was prepared by a method including the following steps:
(1) mixing an aqueous solution of cystamine dihydrochloride with an aqueous solution of KOH for reaction;
(2) extracting with a dichloromethane solution, followed by rotary evaporating and drying to obtain 15.272 g of cystamine in an organic phase, with a yield of an actual product mass/a theoretical product mass=15.272/19.881=76.82%; and
(3) adding a certain amount of cystamine to a three-necked flask, and adding acrylamide in a stoichiometric ratio of the cystamine to the acrylamide of 1.0:2.0, heating to 75° C. and performing reaction for 10 h to obtain the modified cystamine curing agent, recorded as curing agent 2.

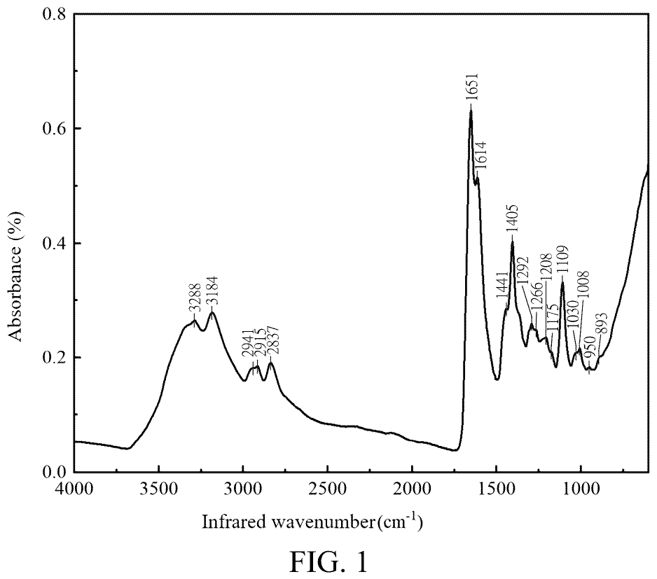

A Fourier transform infrared spectrum of the product is shown in FIG. 1. As can be seen from the FIGURE, the product has a correct structure, and is a target compound.

### Example 3

This Example provides an acrylamide-modified cystamine curing agent, which was prepared by a method including the following steps:
(1) mixing an aqueous solution of cystamine dihydrochloride with an aqueous solution of KOH for reaction;
(2) extracting with a dichloromethane solution, followed by rotary evaporating and drying to obtain 15.272 g of cystamine in an organic phase, with a yield of an actual product mass/a theoretical product mass=15.272/19.881=76.82%; and
(3) adding a certain amount of cystamine to a three-necked flask, and adding acrylamide in a stoichiometric ratio of the cystamine to the acrylamide of 1.0:1.5, heating to 75° C. and performing reaction for 10 h to obtain a mixed curing agent of modified cystamine and the cystamine, recorded as curing agent 3.

The structural formula of the modified cystamine curing agents in Examples 1 and 2 is:

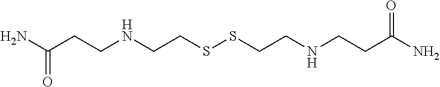 [SMILES: NC(=O)CCNCCSSCCNCCC(N)=O]

The modified cystamine curing agent in Example 3 is a mixture of the cystamine and the acrylamide-modified curing agent.

### Example 4

A method for preparing a solvent-free self-healing polyurethane based on dynamic disulfide bonds was performed, which includes the following steps:
(1) adding a certain amount of polycaprolactone diol 1000 in a four-necked flask, and dehydrating under vacuum at 110° C. for 2 h; and cooling a temperature to 50° C., adding IPDI in a stoichiometric ratio of diisocyanate to dihydric alcohol of 4.0:2.0, and reacting under stirring at 80° C. for 6 h in a nitrogen atmosphere to obtain a transparent isocyanate-terminated polyurethane prepolymer;
(2) reacting the isocyanate-terminated polyurethane prepolymer obtained in the step (1) with glycidol at a molar ratio of 1.0:2.0 to obtain an epoxy-terminated polyurethane prepolymer with a reaction condition of 70° C. for 6 h, where an entire process was performed in the nitrogen atmosphere;
(3) adding the epoxy-terminated polyurethane prepolymer to a curing agent 1 having a molar ratio of reactive functional groups (active hydrogen: epoxy group) of 1.0:1.0, stirring evenly and pouring into a 2 mm thick mold, and hot-pressing and curing at 100° C. for 10 h to obtain the solvent-free self-healing polyurethane based on the dynamic disulfide bonds provided by the present disclosure, recorded as PU1;
(4) adding the epoxy-terminated polyurethane prepolymer to a curing agent 2 with the molar ratio of the reaction functional groups (the active hydrogen: the epoxy group) of 1.0:1.0, stirring evenly and pouring into the 2 mm thick mold, and hot-pressing and curing at 100° C. for 10 h to obtain the solvent-free self-healing polyurethane based on the dynamic disulfide bonds provided by the present disclosure, recorded as PU2; and
(5) adding the epoxy-terminated polyurethane prepolymer to a curing agent 3 with the molar ratio of the reaction functional groups (the active hydrogen: the epoxy group) of 1.0:1.0, stirring evenly and pouring into the 2 mm thick mold, and hot-pressing and curing at 100° C. for 10 h to obtain the solvent-free self-healing polyurethane based on the dynamic disulfide bonds provided by the present disclosure, recorded as PU3.

The solvent-free self-healing polyurethane based on the dynamic disulfide bonds provided by the present disclosure was subjected to tensile strength and elongation at break tests according to requirements of type 2 specimens in the national standard GB/T 528-2009, the type 2 specimens were completely cut and spliced, and the self-healing performance was measured at a certain temperature and time. The results are shown in Table 1, where a self-healing efficiency is calculated according to a following formula:

$$η = \frac{F \text{?}}{F \text{?}}$$

$$\text{?} \text{indicates text missing or illegible when filed}$$

η—a self-healing efficiency; and
F—a tensile strength or an elongation at break.

TABLE 1
Mechanical performance and self-healing performance
results of self-healing polyurethane

| | PU1 | | PU2 | | PU3 | |
|---|---|---|---|---|---|---|
| | tensile | elongation | tensile | elongation | tensile | elongation |
| | strength | at break | strength | at break | strength | at break |
| original mechanical | 9.5 | 474 | 7.0 | 609 | 8.9 | 224 |
| performance/MPa | | | | | | |
| self-healing | 35.3 | 73.4 | 19.0 | 56.2 | 18.9 | 27.9 |
| efficiency for | | | | | | |
| healing 6 h at room | | | | | | |
| temperature/% | | | | | | |
| self-healing | 77.2 | 82.1 | 54.8 | 60.6 | 20.3 | 52.7 |
| efficiency for | | | | | | |
| healing 6 h at | | | | | | |
| 60° C./% | | | | | | |
| self-healing | 93.6 | 86.5 | 81.4 | 71.9 | 23.9 | 55.4 |
| efficiency for | | | | | | |
| healing 6 h at | | | | | | |
| 80° C./% | | | | | | |

It can be seen from Table 1 that the self-healing polyurethane obtained by the method of the present disclosure has excellent self-healing performance, and curing agents with different amine values may be prepared by adjusting the molar ratio of the acrylamide to the cystamine, thereby adjusting the mechanical performance and self-healing performance. The lower the proportion of the acrylamide, the worse the self-healing performance.

### Example 5

A method for preparing a solvent-free self-healing polyurethane based on dynamic disulfide bonds was performed, which includes the following steps:
(1) adding a certain amount of polycaprolactone diol 1000 in a four-necked flask, and dehydrating under vacuum at 110° C. for 2 h; and cooling a temperature to 50° C., adding HMDI in a stoichiometric ratio of diisocyanate to dihydric alcohol of 4.0:2.0, and reacting under stirring at 80° C. for 6 h in a nitrogen atmosphere to obtain a transparent isocyanate-terminated polyurethane prepolymer;
(2) reacting the isocyanate-terminated polyurethane prepolymer obtained in the step (1) with glycidol at a molar ratio of 1.0:2.0 to obtain an epoxy-terminated polyurethane prepolymer with a reaction condition of 70° C. for 4 h, where an entire process was performed in the nitrogen atmosphere; and
(3) adding the epoxy-terminated polyurethane prepolymer to a curing agent 1 having a molar ratio of reactive functional groups (active hydrogen: epoxy group) of 1.0:1.0, stirring evenly and pouring into a 2 mm thick mold, and hot-pressing and curing at 100° C. for 8 h to obtain the solvent-free self-healing polyurethane based on the dynamic disulfide bonds provided by the present disclosure, recorded as PU4.

The solvent-free self-healing polyurethane based on the dynamic disulfide bonds provided by the present disclosure was subjected to tensile strength and elongation at break tests according to requirements of type 2 specimens in the national standard GB/T 528-2009, the type 2 specimens were completely cut and spliced, and the self-healing performance was measured at a certain temperature and time. The results are shown in Table 2, where a self-healing efficiency is calculated according to a following formula:

$$η = \frac{F \text{?}}{F \text{?}}$$

$$\text{?} \text{indicates text missing or illegible when filed}$$

η—a self-healing efficiency; and
F—a tensile strength or an elongation at break.

TABLE 2
Mechanical performance and self-healing performance
results of self-healing polyurethane PU4

| | PU4 | |
|---|---|---|
| | | elongation at |
| | tensile strength | break |
| original mechanical performance/MPa | 9.0 | 452 |
| self-healing efficiency for healing 6 h at | 30.8 | 40.0 |
| room temperature/% | | |
| self-healing efficiency for healing 6 h at | 57.7 | 52.4 |
| 60° C./% | | |
| self-healing efficiency for healing 6 h at | 83.7 | 73.9 |
| 80° C./% | | |

It can be seen from Table 2 that the self-healing polyurethane obtained by the method of the present disclosure has excellent self-healing performance.

The present disclosure has been described in detail above in conjunction with specific implementations and explanatory examples. However, these descriptions should not be construed as limiting the present disclosure. Those skilled in the art will appreciate that, without departing from the spirit and scope of the present disclosure, various equivalent substitutions, modifications or improvements may be made to the technical solutions and implementations of the present disclosure, all of which fall within the scope of the present disclosure. The scope of protection of the present disclosure shall be determined by the appended claims.

The contents not described in detail in the specification of the present disclosure belong to the common knowledge of those skilled in the art.

1. A method for preparing a modified cystamine curing agent, comprising:
obtaining a reaction solution by reacting an aqueous solution of cystamine dihydrochloride with an aqueous solution of KOH;
extracting the reaction solution with a dichloromethane solution, followed by rotary evaporating and drying to obtain cystamine; and
obtaining an acrylamide-modified cystamine curing agent by reacting the cystamine with acrylamide.

2. The method for preparing the modified cystamine curing agent according to claim 1, wherein the cystamine is reacted with the acrylamide at a reaction temperature of 60 to 100° C. for a reaction time of 4 to 10 h, and a molar ratio of the cystamine to the acrylamide is 1.0:1.5 to 1.0:2.1.

3. A modified cystamine curing agent, obtained by the method for preparing the modified cystamine curing agent according to claim 1.

4. A method for preparing a self-healing polyurethane, comprising:
obtaining an isocyanate-terminated polyurethane prepolymer by reacting diisocyanate with a dihydric alcohol;
obtaining an epoxy-terminated polyurethane prepolymer by reacting the isocyanate-terminated polyurethane prepolymer with glycidol; and
obtaining a solvent-free self-healing polyurethane based on dynamic disulfide bonds by reacting the epoxy-terminated polyurethane prepolymer with the modified cystamine curing agent according to claim 3.

5. The method for preparing the self-healing polyurethane according to claim 4, wherein the diisocyanate comprises one or more of toluene diisocyanate TDI, isophorone diisocyanate IPDI, methylene diphenyl diisocyanate MDI, dicyclohexylmethane diisocyanate HMDI or hexamethylene diisocyanate HDI;
the dihydric alcohol comprises one or more of polycaprolactone diol PCL, polypropylene glycol PPG, polyethylene glycol PEG, or polytetramethylene glycol PTMG;
wherein the diisocyanate is reacted with the dihydric alcohol in a molar ratio of the diisocyanate to the dihydric alcohol of 3.0:2.0 to 5.0:2.0.

6. The method for preparing the self-healing polyurethane according to claim 4, wherein reacting the diisocyanate with the dihydric alcohol comprises:
dehydrating the dihydric alcohol by vacuuming at a temperature of 100 to 120° C. for a vacuuming time of 2 to 5 h;
cooling dehydrated dihydric alcohol to a temperature below 60° C.; and
adding diisocyanate to cooled dihydric alcohol for reaction under stirring at 60 to 85° C. for 3 to 6 h under nitrogen protection to obtain the isocyanate-terminated polyurethane prepolymer.

7. The method for preparing the self-healing polyurethane according to claim 4, wherein the isocyanate-terminated polyurethane prepolymer is reacted with glycidol at a reaction temperature of 50 to 85° C. for a reaction time of 2 to 6 h in a nitrogen atmosphere; and a molar ratio of the isocyanate-terminated polyurethane prepolymer to glycidol is 1.0:2.0 to 1.0:2.1.

8. The method for preparing the self-healing polyurethane according to claim 4, wherein a molar ratio of a reactive functional group of the epoxy-terminated polyurethane prepolymer to a reactive functional group of the modified cystamine curing agent is 1.0:0.95 to 1.0:1.05; and
the reactive functional group of the epoxy-terminated polyurethane prepolymer is active hydrogen, and the reactive functional group of the modified cystamine curing agent is epoxy group.

9. The method for preparing the self-healing polyurethane according to claim 4, wherein the epoxy-terminated polyurethane prepolymer is reacted with the modified cystamine curing agent at a reaction temperature of 80 to 130° C. for a reaction time of 6 to 24 h.

10. A self-healing polyurethane, obtained by the method for preparing the self-healing polyurethane according to claim 4.
